(19) United States
(12) Patent Application Publication
(10) Pub. No.: US 2010/0173003 A1
(43) Pub. Date: Jul. 8, 2010

(54) COMPOSITION AND METHOD FOR ENCAPSULATING BENEFIT AGENTS

(75) Inventors: Ashoke K. SenGupta, Barrington, IL (US); Dave Kotloski, Carpentersville, IL (US); Vincent J. Losacco, Geneva, IL (US); David McGregor, Grayslake, IL (US)

Correspondence Address: MARSHALL, GERSTEIN & BORUN LLP, 233 SOUTH WACKER DRIVE, 6300 WILLIS TOWER, CHICAGO, IL, 60606-6357, US

(73) Assignee: Amcol International, Hoffman Estates, IL (US)

(21) Appl. No.: 12/601,686

(22) PCT Filed: May 27, 2008
(86) PCT No.: PCT/US08/64884

§ 371 Date: Feb. 1, 2010

Related U.S. Application Data
(60) Provisional application No. 60/939,988, filed on May 24, 2007.

(51) Int. Cl. A61K 9/16

(52) U.S. Cl. 424/497

(57) ABSTRACT

A benefit agent encapsulated in a particulate-based encapsulant, and a method of manufacturing the encapsulated benefit agent, are disclosed.

### BACKGROUND

Numerous product formulations rely on their “active” ingredients (referred to herein as benefit agent) insofar as providing for functional benefits. Examples of such products include various personal care, cosmetic, pharmaceutical, nutraceutical, agrochemical, household, and pet product formulations. The formulations can be oil-in-water (O/W) emulsions or water-in-oil (W/O) emulsions or simply water-based or oil-based or solid compositions.

In formulating benefit agent-laden products, needs arise for incorporating active ingredients into formulations in encapsulated forms. The reasons may include: i) degradation of a benefit agent when exposed to the formulation conditions; ii) it is intended that the active releases slowly in delivering a benefit; and iii) it is desirable that the actives' beneficial effects are manifested only during product application and not during product storage. Under these circumstances, having an encapsulated active ensures that it is available to deliver its benefit in the most desirable fashion.

The present invention relates to methods and compositions for encapsulating benefit agents within a particulate matrix comprising a smectite clay mineral, a coagulating agent, a water-soluble polymeric flocculant, and a water-insoluble copolymer. The particulate-based encapsulant allows the benefit agent to be released under shear, attrition, and compression forces being applied during product application (for example, via brushing, scrubbing, rubbing, wetting).

### SUMMARY OF THE INVENTION

The present invention discloses methods and compositions for encapsulating benefit agents, which do not rely on the use of cross-linked polymers, porous cross-linked polymers, and/or a polymeric shell formed by coacervation of polymers, unlike the methods known in the art. Rather, the active ingredient is entrapped within a particulate matrix comprising in part of flocculated particles of a smectite clay mineral. The flocculated particles, containing a benefit agent, in turn remain embedded within a composite material comprising a hydrophobic, water-insoluble copolymer and a smectite clay mineral.

According to an embodiment of the present invention, the benefit agent can be either a water-insoluble, particulate material, or a water-soluble material. While dispersed in water in a particulate form (referred to herein as Particulate 1), the benefit agent is first coagulated with a particulate material (referred to herein as Particulate 2), namely, an inorganic or an organic solid or liquid that meets certain specifications for surface charge, particle size, and aspect ratio. Alternatively, if the active is soluble in water, it is first adsorbed onto a particulate material of the foregoing type, utilizing electrostatic attraction and/or hydrogen bonding interactions between the active and the particulate material.

Both Particulate 1 and Particulate 2 are preferably sheared individually or in a mixture in aqueous suspensions prior to being subjected to aqueous solution conditions under which they undergo coagulation. Such hetero-coagulation (coagulation between dissimilar particulate materials) may involve intermediate steps of homo-coagulation (coagulation between similar particulate materials) between the individual particles of Particulate 1, as well as between the individual particles of Particulate 2, wherein the homo-coagulated particles of the two particulate materials subsequently undergo hetero-coagulation to form the mixed coagulum (coagulated mass/particles) of Particulate 1 and Particulate 2.

The resulting mixed coagulum is subsequently treated (in an aqueous suspension) with at least one high molecular weight (weight average molecular weight >500,000 Dalton) polymeric flocculant, wherein the coagulum particles grow in size under the flocculating influence of the polymer. The resulting flocculated particles (flocs), with a particle size typically in the range 200-50,000 micron, is processed further, albeit involving no chemical reaction or polymer coacervation, to produce a dry (with a volatile material content of less than 20% by weight), particulate-based encapsulant, comprising the said coagulum with a surface-coating of one or any combination of the following types of materials:
i) hydrophilic polymer
ii) hydrophobic polymer
iii) amphiphilic copolymer
iv) composite material comprising a particulate material (e.g., smectite clay) and a polymer
v) wax.

Accordingly, the most preferred method of producing the foregoing particulate-based encapsulant for a benefit agent, involves the following steps, depending on whether the active material is water-dispersible or water-soluble.

Water-Dispersible Active
i) Shearing the water-dispersible active, Particulate 1, in a water-based dispersion.
ii) Shearing Particulate 2 in a water-based dispersion either together with Particulate 1 or separately.
iii) Coagulation of Particulate 1 with Particulate 2 from a mixed aqueous dispersion of the two particulate materials, using coagulating agents known in the art. An alternative method of coagulating the two particulate materials involves mixing them in a polar solvent (preferably water), wherein the electrical charge of Particulate 1 surface is opposite in sign to that of Particulate 2 surface. One embodiment of this method requires that, prior to coagulation, the two particulate materials individually are treated with ionic surfactants or ionic polymers to render them oppositely charged; for example, Particulate 1 is treated with an anionic surfactant and Particulate 2 with a cationic surfactant.
iv) Flocculation of the coagulum particles, using a polymeric flocculating agent, resulting in flocculated particles with a particle size in the range of 0.1-50,000 micron. The polymeric flocculating agent may be selected from the group comprising of high molecular weight (i.e., molecular weight >500,000 Dalton) nonionic, anionic, and cationic polymers, and mixtures thereof.
v) Separation and dewatering of the flocculated coagulum, involving, for example, operations such as sedimentation, decanting, filtration, and centrifugation. The volatile material (primarily water) content of the separated flocculated particles is in the range of 75-98% by weight.
vi) Mixing the flocculated coagulum with a water-based coating material for surface-coating the coagulum solids.
vii) Drying the resulting coagulum-coating material mixture to a moisture/volatile material content of less than 20% by weight.

Water-Soluble Active
i) Dissolving the water-soluble active in water.
ii) Shearing the foregoing Particulate 2 in a water-based dispersion.
iii) Adsorbing the water-soluble active onto the surface of Particulate 2, by slowly adding, for example, the solution from (i) to the sheared dispersion from (ii), and subsequently mixing the resulting dispersion under low-shear agitation.
iv) Upon adsorbing a water-soluble active onto the surface of particles of Particulate 2, coagulating the particles of Particulate 2, using coagulating agents known in the art. According to an embodiment, the benefit agent itself could serve as a coagulating agent for Particulate 2, requiring that the sign of the electrical charge (anionic or cationic) of the benefit agent is opposite to that of the surface charge of Particulate 2, wherein electrostatic attraction-driven adsorption of the benefit agent onto the surface of Particulate 2 leads to the coagulation of Particulate 2.
v) Flocculation of the coagulum particles, using a polymeric flocculating agent, resulting in flocculated particles with a particle size in the range of 0.1-50,000 micron. The polymeric flocculating agent may be selected from the group comprising of high molecular weight (i.e., molecular weight >500,000 Dalton) nonionic, anionic, and cationic polymers, and mixtures thereof.
vi) Separation and dewatering of the flocculated coagulum, involving, for example, operations such as sedimentation, decanting, filtration, and centrifugation. The volatile material (primarily water) content of the separated flocculated particles is in the range of 75-98% by weight.
vii) Mixing the flocculated coagulum with a water-based coating material for surface-coating the coagulum solids.
viii) Drying the resulting coagulum-coating material mixture to a moisture/volatile material content of less than 20% by weight.

According to the most preferred embodiment of the present invention, a water-dispersible benefit agent, i.e., Particulate 1, has a mean primary particle size of less than 10 microns, Particulate 2 is a smectite clay mineral, preferably montmorillonite, and the water-based coating material is a composite material comprising (water-free basis) a water-insoluble copolymer and a smectite clay mineral. The mean particle size of the smectite clay mineral, when the clay particles are sheared in de-ionized water, is less than 50 microns.

An object of the present invention is to produce the encapsulated benefit agent in the form of a particulate material having a mean particle size in the range of about 200-10,000 microns. Producing relatively large-sized particulate materials (i.e., 200-10,000 microns in size), using particulate material components that are much smaller in size (for example, a smectite clay mineral, and a benefit agent smaller than 10 microns in size), is rather difficult and invariably requires expensive processing steps. By entrapping a benefit agent within a matrix of flocculated particles of a smectite clay mineral, as per the methods and compositions disclosed in the present invention, it is now possible to avoid energy and labor-intensive processes of producing large-sized particulate materials, serving as an encapsulant for benefit agents, from relatively small-sized particulate components. Also, the use of a relatively inexpensive material such as a smectite clay mineral, as the primary component for a benefit agent encapsulant, as per the methods and compositions of the present invention, leads to the disclosure of an encapsulation system for benefit agents, that is considerably cheaper to produce, as compared to the encapsulation systems known in the art involving cross-linked and/or coacervated polymers.

### DETAILED DESCRIPTION OF THE INVENTION

The more detailed specifications for the inventive composition and methods are given below.

I. Pre-Coagulation Forms for Particulate 1 and Particulate 2

Prior to coagulation with Particulate 2, Particulate 1 remains essentially in any of following forms:
i) water-insoluble particles having a particle size of preferably <10 micron, more preferably <1 micron, and most preferably <0.1 micron, once the particles are sheared in water or a polar organic liquid to form a dispersion;
ii) particulate dispersion in an organic liquid (resulting upon shearing the particulate material in an organic liquid), wherein the particle size in the dispersed state is preferably <10 microns, more preferably <1 micron, and most preferably <0.1 micron; and
iii) native form of a water-insoluble material.

In producing the aforementioned dispersions of a benefit agent, dispersing agents known in the art may be used to facilitate shear-induced dispersion of Particulate 1 in a dispersion medium selected from water or an organic solvent. Non-limiting examples of dispersants for water- and polar organic solvent-based dispersions include various polyacrylates, polysulfonates, polyphosphates, polysulfates, polyalcohols, polyglycols, polyethylene oxides, and water-soluble/dispersible surfactants selected from anionic, cationic, non-ionic, and zwitterionic surfactants and amphiphilic copolymers. The dispersing agents suitable for non-polar organic solvents include, but not limited to, oil-soluble/dispersible polymers (e.g., polyhydroxystearate) and amphiphilic copolymers (e.g., polyethylene glycol 30-dipolyhydroxystearate, silicone copolymers) as well as mono- and di-alkyl/alkyl-aryl surfactants having a hydrocarbon chain length of >C₈.

Prior to coagulation with Particulate 1, Particulate 2 can remain in any of the following forms:
i) water-insoluble particles having a mean particle size of preferably <50 microns, more preferably <5 micron, and most preferably <1 micron, once the particles are sheared in water or a polar organic liquid to form a dispersion;
ii) co-dispersed with Particulate 1 in water or a polar organic liquid;
iii) particulate dispersion in an organic liquid (resulting upon shearing the particulate material in an organic liquid), wherein the particle size in the dispersed state is preferably <50 microns, more preferably <5 micron, and most preferably <1 micron; and
iv) native form of a water-insoluble material.

Dispersing agents such as the ones noted above could be used in producing the foregoing dispersions of Particulate 2.

In order to be fully useful for the present invention, Particulate 2, in its native form, i.e., without any surface-modification, preferably meets any of the following specifications:
i) the particle surface charge is anionic in the pH range of 1-5;
ii) the particle surface charge is cationic in the pH range of 3-9; and
iii) the particles have an aspect ratio in the range of 100-2000, wherein the aspect ratio is defined as the ratio of the longest to the shortest dimension of a particulate material.

According to the most preferred embodiment of the present invention, Particulate 2 is a smectite clay mineral.

II. Coagulation/Flocculation of Particulate 1 and Particulate 2

According to the present invention, Particulate 1-to-Particulate 2 coagulation/flocculation may be carried out based on any coagulation/flocculation mechanisms known in the art, including the following:
i) charge neutralization, wherein electrically charged particles coagulate under the domineering influence of van der Waals forces acting between the particles, upon neutralization of the particle surface charge due to the adsorption of an oppositely charged moiety (ionic surfactants and polymers, simple ions, oppositely charged particles) on the particle surface;
ii) patch coagulation involving sticky collision, say, between the anionic portion of surface of an anionic particle and any “cationic patch” developed on the surface of another anionic particle due to the localized adsorption of an oppositely charged polymer onto the particle surface;
iii) coagulation of dispersed particles under the influence of polymers adsorbed on the particle surface, upon instilling conditions that turn the dispersion medium into a bad solvent for the adsorbed polymer; and
iv) bridging flocculation of dispersed particles by a polymer chain that concurrently adsorbs on more than one particle.

According to the present invention, a preferred method for effecting coagulation, in a manner suitable for producing the particulate-based encapsulant disclosed herein, involves the following steps:
i) shearing a sodium smectite clay in water;
ii) shearing a calcium smectite clay in water;
iii) combining the foregoing clay suspensions under agitation, with a weight ratio of 1:1 for the sodium smectite to the calcium smectite;
iv) diluting the mixed clay suspensions with deionized water;
v) shearing Particulate 1 (benefit agent) in water to form a dispersion, using a cationic surfactant (e.g., cetylpyridinium chloride, quaternary ammonium compounds) as a dispersing agent;
vi) adding the above pre-sheared suspension of Particulate 1 to the foregoing dilute suspension of the smectite clays, under gentle agitation, leading to partial coagulation of Particulate 1 with Particulate 2;
vii) adding an aliquot of an aqueous solution of the aforementioned cationic surfactant to the mixed suspension from step (vi), leading to complete coagulation of Particulate 1 with Particulate 2, with a clear layer of water separating from a layer of Particulate 1-Particulate 2 coagulum;
viii) adding an aliquot of a dilute aqueous solution of a ultra high molecular weight (weight average molecular weight >5 million Dalton) anionic polymer (sodium acrylate-acrylamide copolymer) to the suspension from step (vii), under gentle agitation;
ix) diluting the suspension with deionized water;
x) adding an aliquot of a dilute aqueous solution of a high molecular weight cationic polymer (cationic guar gum) having a relatively low cationic charge, under gentle agitation, leading to heavy flocculation of the suspension;
xi) shearing the resulting flocculated mass into much smaller floc particles;
xii) repeating steps (viii) and (x) sequentially, adding additional amounts of the two polymers, and repeating the flocculation process; and
xiii) shearing the resulting floc particles to a smaller size of about 0.1-1 cm in size.

Yet another preferred method of producing Particulate 1-Particulate 2 flocs, involves the following steps:

i) shearing Particulate 1 (benefit agent) in water to form a dispersion, using an anionic surfactant as a dispersing agent;

ii) diluting the above dispersion with deionized water;

iii) adding an aliquot of an aqueous solution of alum to the above dispersion under agitation;

iv) shearing a sodium smectite clay in water and adding the pre-sheared suspension to the above dispersion under agitation;

v) adding a cationic polymer (cationic guar gum) to the dispersion under gentle agitation;

vi) adding an anionic polymer (xanthan gum) and/or an anionic particle (smectite clay) to the dispersion, upon which heavily flocculated chunks of coagulated particulate materials start to appear;

vii) shearing the flocs to a smaller size;

viii) adding an additional amount of the cationic polymer, upon which the flocs grow in size;

ix) repeating steps (vi) through (viii), until the flocs appear to be fairly rigid and about 0.1-1 cm in size.

Yet another preferred method used currently for the coagulation process is similar to the above except that subsequent to step (iv), a sodium acrylate-acrylamide copolymer (Magnafloc 115 or Magnafloc TD 25 from Ciba Specialty Chemicals), rather than cationic guar gum and xanthan gum, is added intermittently to the dispersion in several portions, with the step of shearing the flocs to a smaller size carried out in between the addition of the copolymer portions.

III. Separation and Dewatering of Coagulum

The coagulum prepared in accordance with a preferred method described above is allowed to settle for a certain period of time, leading to the separation of a clear layer of water from a layer of coagulum-sludge. After decanting out the separated layer of water, the coagulum-sludge is further dewatered using a filtration method. The solids-content of the dewatered coagulum-sludge is in the range of 1-20% by weight.

IV. Dispersion of Coagulum in a Coating Solution/Suspension

The dewatered coagulum/floc is dispersed in a solution/suspension of a coating material, while maintaining a ratio of 0.1-10 for the relative weight (dry-basis) of the coagulum to the weight (dry-basis) of the coating material. Vigorous, yet, low-shear, agitation is used for this dispersion process. The polymeric coating materials useful for the present invention include, but not limited to, the natural film-forming polymers selected from cellulose and its derivatives, various film-forming proteins and their derivatives, chitosan and its various derivatives, starch and modified starch, and various natural gum polymers and their derivatives, polyvinyl alcohol, polymers and copolymers of vinyl pyrrolidone, polymers and copolymers of acrylic acid, polymers and copolymers of methacrylic acid, amphiphilic copolymers such as polyethylene glycol 30-dipolyhydroxystearate, various silicone polymers and copolymers, and polyurethane and its derivatives. According to the most preferred embodiment of the present invention, the coating material comprises a polymer and a particulate filler material selected from the group consisting of, but not limited to, a smectite clay mineral including organo-modified smectite clays, kaolin, talc, titanium dioxide, zinc oxide, alumina, silica, cerium oxide, mica, calcium carbonate pigment, latex, and mixtures thereof. The amount of the particulate filler material in the coating material can be 0-95% by weight of the total weight of the coating material (dry-basis).

V. Drying of the Coagulum Dispersion

The coagulum-coating material dispersion is dried to a volatile matter content of less than 20% by weight, using any of the methods known in the art.

### Example I

This example describes an application wherein a benefit agent encapsulated in a particulate-based encapsulant of the present invention, demonstrated its intended benefit, when included in a toothpaste formulation.

The benefit agent is a water-insoluble, blue-colored pigment (copper phthalocyanate), and its intended use is for the toothpaste-froth to show a progressively increasing intensity of blue color with passage of time during brushing of teeth. The encapsulated form in which the pigment was included in the toothpaste formulation was derived in accordance with the various methods described above, wherein Particulate 2 was a sodium smectite clay, coagulated with the pigment in accordance with a preferred method described in section II. The coating polymer used was hydroxypropylmethyl cellulose available under the tradename, Methocell, from Dow Chemical Company. The various composition parameters for the encapsulated form of the said benefit agent are given below.

TABLE I
Pigment Dispersion (Sheared with a dispersion blade agitator)

| | Pigment, | Sodium Lauryl | Deionized Water, |
|---|---|---|---|
| Dispersion | Weight % | Sulfate, Weight % | Weight % |
| Copper | 10 | 0.2 | 89.8 |
| Phthalocyanate | | | |
| (Supplier: Keystone | | | |
| sold under the | | | |
| Keyplast tradename) | | | |

TABLE II
Smectite Clay Dispersion

| | Clay, | Deionized Water, | Method of |
|---|---|---|---|
| Dispersion | Weight % | Weight % | Shearing |
| Sodium Smectite | 5 | 95 | Dispersion Blade |
| | | | Agitator |

TABLE III
Coagulation Composition

| | Pigment | 35% Alum | Smectite Clay | 0.4% Cationic | 2% Xanthan Gum | Water + Sodium |
|---|---|---|---|---|---|---|
| | Suspension, | Solution, | suspension, | Guar Gum Solution, | Solution, | Hydroxide for pH |
| Batch | Weight % | Weight % | Weight % | Weight % | Weight % | Adjustment, weight % |
| 1 | 6.98 | 0.31 | 13.26 | 16.57 | 3.31 | 59.57 |
| 2 | 6.04 | 0.33 | 12.08 | 12.08 | 3.02 | 66.45 |

TABLE IV
Coagulum Dispersion in Hydroxypropylmethyl Cellulose
(HPMC)

| | 4% HPMC | | Method of |
|---|---|---|---|
| Batch | Solution, Weight % | Coagulum, Weight % | Dispersion |
| 1 | 71 | 29 | Heat HPMC to |
| | | (12.85 wt. % solids) | 60° C., add |
| | | | coagulum, mix |
| | | | with propeller |
| | | | agitator |
| 2 | 76.1 | 23.9 | Heat HPMC to |
| | | | 60° C., add |
| | | | coagulum, mix |
| | | | with propeller |
| | | | agitator |

Both the batches in Table IV were dried in an oil bath (canola oil) using a weight ratio of about 10:1 (about 500 g dispersion to 5,000 g oil) for oil to coagulum dispersion. The dispersion was added to the oil bath at 45° C. under vigorous agitation, after which the temperature was increased slowly to 95° C. and subsequently the bath was maintained at that temperature for about 3 hours to complete the drying process. The dried solids was filtered using a 200 micron mesh filter after which the filter cake was rinsed with heptane, and the resulting solids were dried to a residual volatile content of about 1% by weight. The dried solids were in the form of free-flowing particles in the size range of about 200-850 micron.

The encapsulated pigment thus obtained (Batch 1) was included in a toothpaste formulation received from a commercial manufacturer, at a dosage of about 0.42% (i.e., about 0.1% pigment by weight). About 1.5 g of the toothpaste and 0.5 g of water were weighed out on a glazed ceramic plate. The resulting diluted toothpaste was massaged against the ceramic plate, using gentle brushing strokes of a toothbrush. The froth collected after 0.5 minute, 1 minute, and 2 minutes of brushing was analyzed using a color meter. The “b” values, indicating the intensity of blue color, increased from −5.34 after 0.5 minute of brushing to about −11.63 after 1 minute of brushing to about −19.24 after 2 minutes of brushing (against a target value of −15 after 2 minutes of brushing).

### Example II

This example describes an application wherein multiple benefit agents were included in a toothpaste formulation, using the particulate-based encapsulant of the present invention.

One of the benefit agents is a water-insoluble, blue-colored pigment (copper phthalocyanate), and its intended use is for the toothpaste-froth to show a progressively increasing intensity of blue color with passage of time during brushing of teeth. The other benefit agent is cetylpyridinium chloride (CPC), a quaternary ammonium compound-based cationic surfactant that can function as an antigingivitis agent. The particulate-based encapsulant was derived in accordance with the various methods described above, wherein Particulate 2 was a 1:1 (weight-basis) mixture of a sodium smectite clay and a calcium smectite clay, coagulated with the pigment in accordance with the most preferred method described in section II, wherein the aforementioned cationic surfactant, CPC, was used as a coagulating agent. The coating material used was an aqueous suspension of the foregoing sodium smectite, which contained an amphiphilic copolymer, polyethylene glycol 30-dipolyhydroxystearate (PEG (30) Dipolyhydroxystearate), as the surface-modifier for the clay. The amount of the amphiphilic copolymer was about 100%, based on the weight of sodium smectite. The coagulum and the coating material were mixed under vigorous agitation. The weight-ratio (dry-basis) of coagulum to the coating material was varied in the range of 1:1-1.38:1. The various composition parameters for the encapsulated form of the said benefit agents are given below.

TABLE V
Pigment Dispersion (Sheared with a dispersion blade agitator)

| | Pigment, | CPC, | Deionized Water, |
|---|---|---|---|
| Dispersion | Weight % | Weight % | Weight % |
| Copper | 10 | 0.7 | 89.3 |
| phthalocyanate | | | |
| (Supplier: Ciba) | | | |

TABLE VI
Smectite Clay Dispersion

| | Clay, | Deionized | |
|---|---|---|---|
| | Weight | Water, | |
| Dispersion | % | Weight % | Method of Shearing |
| 1 - Sodium Smectite | 2.3 | 97.7 | Dispersion Blade Agitator |
| 2 - Calcium Smectite | 4 | 96 | Dispersion Blade Agitator |

TABLE VII
Coagulation/Flocculation Composition

| Sodium | Calcium | | | Sodium Polyacrylate- | Cationic | |
|---|---|---|---|---|---|---|
| Smectite, | Smectite, | Pigment, | CPC, | Acrylamide | Guar, | Water, |
| Weight % | Weight % | Weight % | Weight % | Copolymer, Weight % | Weight % | weight % |
| 0.2286 | 0.2286 | 0.1143 | 0.0476 | 0.0169 | 0.0201 | 99.3439 |

TABLE VII
Coating Material Composition

| Sodium | Polyethylene Glycol 30 − | Water + | |
|---|---|---|---|
| Smectite, | dipolyhydroxystearate, | Preservative, | Method of |
| Weight % | Weight % | Weight % | Shearing |
| 5 | 5 | 90 | Rotor-stator |
| | | | homogenizer |

TABLE VIII
Coagulum Dispersion in the Coating Material

| | Coating Material, | | Water, |
|---|---|---|---|
| Batch | Weight % | Coagulum, Weight % | Weight % |
| 1 | 28.571 (10.3 wt. % solids) | 57.143 (7.1 wt. % solids) | 14.286 |
| 2 | 32.911 (10.3 wt. % solids) | 52.743 (6.4 wt. % solids) | 14.346 |

Both the batches in Table VIII were dried in an oven set at 110° C. to a moisture-content of <2% by weight. The dried material was milled and subsequently sieved to a size in the range of 300-600 microns. The encapsulated pigment thus obtained was included in a toothpaste formulation received from a commercial manufacturer, at a dosage corresponding to about 0.1% and 0.086% by weight of pigment, respectively, for Batch 1 and Batch 2. About 1.5 g of the toothpaste and 0.25 g of water were weighed out on a ceramic plate. The resulting diluted toothpaste was massaged against the ceramic plate, using gentle brushing strokes of a toothbrush. The froth collected after 0.5 minute, 1 minute, and 2 minutes of brushing was analyzed using a color meter. The results for the “b” values, indicating the intensity of blue color are shown in Table IX. In order to demonstrate the benefits of the present invention over an encapsulation method used in the prior art, Table IX also includes the “b” value results for the prior-art encapsulation method.

TABLE IX
Color Intensity Test results

| Encapsulant | Time of Brushing, minute | B value |
|---|---|---|
| Present Invention, Batch 1 | 0.5 | −18.71 |
| | 1 | −25.25 |
| | 2 | −27.68 |
| Present Invention, Batch 2 | 0.5 | −20.04 |
| | 1 | −23.60 |
| | 2 | −27.37 |
| Prior Art Encapsulant - Ciba | 0.5 | −10.64 |
| Pigment | 1 | −14.18 |
| | 2 | −16.38 |

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. A composition containing a benefit agent encapsulated in a particulate-based encapsulant having a mean particle size in the range of 200-5,000 microns while containing less than 20% by weight of water and other volatile materials, the said encapsulant comprising:
(i) the benefit agent;
(ii) a smectite clay mineral;
(iii) a coagulating agent;
(iv) a flocculating agent; and
(v) a composite material comprising a hydrophobic, water-insoluble copolymer and a smectite clay mineral, with (i) through (iv) forming flocculated particulate materials upon mixing, that are embedded in or surface-coated by the composite material (v).

16. The composition of claim 1 wherein the benefit agent is either a water-dispersible material or a water-soluble material selected from the group consisting of pigment, ester, hydrocarbon, silicone, drug, human nutrient, plant nutrient, weed-killer, vitamin, antioxidant, anti-acne agent, skin-lightening agent, sliming agent, tooth-whitening agent, hair conditioning agent, anti-dandruff agent, antimicrobial agent, skin-moisturizing agent, sunscreen active, skin-tanning agent, skin-cooling agent, surface-cleaning agent, polishing agent and mixtures thereof.

17. The composition of claim 1 wherein the smectite clay mineral is montmorillonite.

18. The composition of claim 1 wherein the coagulating agent is selected from the group consisting of tri-, di-, and mono-valent salts of alkali and alkaline earth metals, quaternary ammonium compounds, cationic surfactants and polymers, polyamines with a nitrogen group content of at least 5% by weight, and mixtures thereof.

19. The composition of claim 1 wherein the flocculating agent is selected from the group consisting of anionic, cationic, and non-ionic polymers having a weight average molecular weight of more than 500,000 Dalton.

20. The composition of claim 1 wherein the hydrophobic, water-insoluble copolymer is an amphiphilic copolymer.

21. The composition of claim 6 wherein the amphiphilic copolymer is polyethyleneglycol 30-dipolyhydroxystearate.

22. The composition of claim 1 wherein the ratio of the weight of the hydrophobic, water-insoluble copolymer to the weight of the smectite clay in component (v) is in the range of 0.1-1.

23. The composition of claim 1 wherein the ratio of the total weight of components (i) through (v) to the total weight of component (v) on dry-basis is about 0.1-10.

24. The composition of claim 1 wherein the encapsulant for the benefit agent further contains a surface-coating material as an integral part of the particulate-based encapsulant, at an amount in the range of 10-80% of the total weight of the encapsulant (dry-basis).

25. The composition of claim 10 wherein the surface-coating material is selected from the group consisting of hydrophilic polymer, hydrophobic polymer, amphiphilic copolymer, composite material comprising a particulate material and a polymer, and wax.

26. The composition of claim 11 wherein the surface-coating material is selected from the group consisting of natural film-forming polymers selected from cellulose and its derivatives, film-forming proteins and their derivatives, chitosan and its derivatives, starch and modified starch, natural gum polymers and their derivatives, polyvinyl alcohol, polymers and copolymers of vinyl pyrrolidone, polymers and copolymers of acrylic acid, polymers and copolymers methacrylic acid, amphiphilic copolymers, silicone polymers and copolymers, and polyurethane and its derivatives.

27. The composition of claim 12 wherein the amphiphilic copolymer comprises polyethyleneglycol 30-dipolyhydroxystearate.

28. The composition of claim 10 wherein the surface-coating material includes a particulate filter material at an amount of 0-95% of the total weight of the surface-coating material.

29. The composition of claim 1 further containing a detersive surfactant selected from the group consisting of anionic, nonionic, cationic, and zwitterionic surfactants.
